(19) United States
(12) Patent Application Publication
(10) Pub. No.: US 2025/0360019 A1
(43) Pub. Date: Nov. 27, 2025

(54) Stabilization Assembly and Method of Use

(71) Applicant: Pro med instruments GmbH, Freiburg im Breisgau (DE)

(72) Inventors: Severin Moosmann, Friesenheim (DE); Jan Fiege, Hamburg (DE)

(21) Appl. No.: 19/217,013

(22) Filed: May 23, 2025

Related U.S. Application Data
(60) Provisional application No. 63/650,912, filed on May 23, 2024.

(51) Int. Cl. A61F 5/37

(52) U.S. Cl. CPC A61F 5/3707

(57) ABSTRACT

A stabilization assembly includes a receptacle, a pin, and a retainer. The pin is positioned within the receptacle and the retainer selectively retains the pin in position within the receptacle. Additionally, the receptacle includes a sloped surface surrounding an opening within the receptacle where the pin is received, and the pin includes a corresponding sloped surface for contact with the sloped surface surrounding the opening within the receptacle. A pin can be keyed to a specific receptacle and retainer such that pins of differing geometries will only properly fit with their intended receptacle and retainer. This provides a poka-yoke configuration so users cannot incorrectly associate pins of different styles with the wrong receptacles. For instance, if pins are specifically configured to be received by a specific receptacle, the poka-yoke configuration mentioned here ensures that the pins are installed in the proper stabilization assembly.

### PRIORITY

This application claims priority to and is a nonprovisional of U.S. provisional patent application 63/650,912, entitled “Stabilization Assembly and Method of Use,” filed May 23, 2024, which is incorporated by reference herein.

### BACKGROUND

In certain medical procedures stabilization equipment or devices are used to immobilize a patient or portion of a patient. With certain such devices, there is a stabilization assembly that includes a contact feature or stabilization feature, which contacts the patient. An example of such a stabilization feature is a pin or a pad. The stabilization assembly also includes a holding portion or receptacle, which selectively receives the stabilization feature. In a specific example related to neurosurgery, the stabilization device can be a head fixation device in the form of a skull clamp. While a variety of medical devices and accessories for medical devices have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a perspective view of an exemplary stabilization device in the form of a skull clamp.

FIG. 2 depicts a side view of a stabilization assembly of FIG. 1.

FIG. 3 depicts a side view of another stabilization assembly of FIG. 1.

FIG. 4 depicts a side cross section view of the stabilization assembly of FIG. 2 taken along line 4-4 of FIG. 1.

FIG. 5 depicts a cross section view of the stabilization assembly of FIG. 2 taken along line 5-5 of FIG. 1.

FIG. 6A depicts a cross section view of the stabilization assembly of FIG. 2 taken along line 6-6 of FIG. 2 and shown with the pin outside of the receptacle with the retainer in a closed position.

FIG. 6B depicts a cross section view of the stabilization assembly of FIG. 2 taken along line 6-6 of FIG. 2 and shown with the pin biasing the retainer into an open position.

FIG. 6C depicts a cross section view of the stabilization assembly of FIG. 2 taken along line 6-6 and shown with the pin engaged or coupled to the receptacle with the retainer in the closed position.

FIG. 6D depicts a cross section view of the stabilization assembly of FIG. 2 taken along line 6-6 of FIG. 2 and shown with the pin disengaged or uncoupled in the receptacle with the retainer in the open position.

FIG. 7 depicts a cross section view of the stabilization assembly of FIG. 3 taken along line 7-7 of FIG. 1.

FIG. 8 depicts a cross section view of the stabilization assembly of FIG. 3 taken along line 8-8 of FIG. 7.

FIG. 9 depicts a side view of an alternate retainer usable with the stabilization assemblies of FIG. 2 and/or FIG. 3.

FIG. 10 depicts a perspective view of an exemplary stabilization device in the form of a skull clamp.

FIG. 11 depicts a side view of a stabilization assembly of FIG. 10.

FIG. 12 depicts a side view of another stabilization assembly of FIG. 10.

FIG. 13A depicts a side cross section view of the stabilization assembly of FIG. 11 taken along line 13-13 of FIG. 10.

FIG. 13B depicts a cross section view of the stabilization assembly of FIG. 11 taken along line 13-13 of FIG. 10 and shown with the pin biasing the retainer into an open position.

FIG. 13C depicts a cross section view of the stabilization assembly of FIG. 11 taken along line 13-13 of FIG. 10 and shown with the pin disengaged being removed from the receptacle or being inserted into the receptacle.

FIG. 14 depicts a cross section view of the stabilization assembly of FIG. 11 taken along line 14-14 of FIG. 10.

FIG. 15A depicts a cross section view of the stabilization assembly of FIG. 12 taken along line 15-15 of FIG. 10.

FIG. 15B depicts a cross section view of the stabilization assembly of FIG. 12 taken along line 15-15 of FIG. 10 and shown with the pin biasing the retainer into an open position.

FIG. 15C depicts a cross section view of the stabilization assembly of FIG. 12 taken along line 15-15 of FIG. 10 and shown with the pin disengaged being removed from the receptacle or being inserted into the receptacle.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

With conventional stabilization assemblies, e.g., pin assemblies, and the stabilization features, e.g., pins, there is a lack of feedback provided to the user such that it can be difficult for the user to know with certainty that the stabilization feature, e.g., pin or pad, is properly engaged with the holding portion or receptacle of the stabilization assembly. Note that in certain passages herein the term “pin mount” is used interchangeably with terms like “holding portion” and/or “receptacle.” Also, with certain procedures that are navigation guided, it is desirable to have limited clearance or play between the stabilization feature, e.g., pin, and the bore of the holding portion or receptacle during parts of the stabilization procedure. The embodiments described herein address these and other shortcomings of the conventional stabilization assemblies.

### A. Exemplary Stabilization Assembly

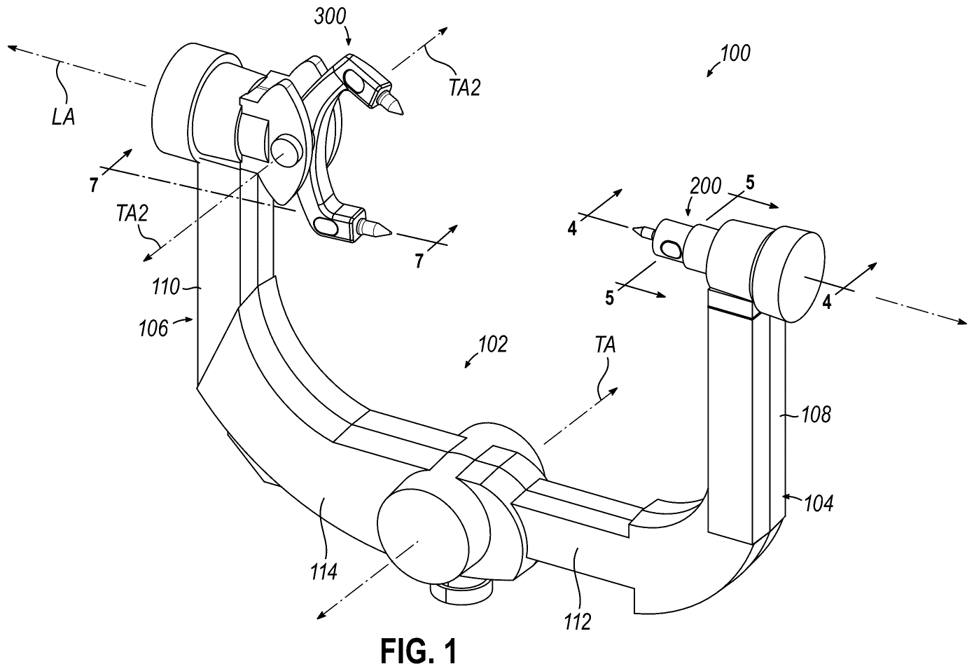

FIG. 1 illustrates an exemplary stabilization device in the form of a skull clamp (100). The skull clamp (100) includes a frame (102) comprised of a pair of arms (104, 106). The arms (104, 106) are moveable relative to one another to adjust a width or opening between upright portions (108, 110) of the arms (104, 106). A base of the frame (102) is comprised of lateral portions (112, 114) of the respective arms (104, 106). The skull clamp (100) is connectable with other equipment directly or indirectly such as a base unit (not shown) that connects with an operating table (not shown). As shown, the skull clamp (100) also includes stabilization assemblies (200, 300), each located at the upper portion of one of the arms (104, 106). Stabilization assembly (300) is configured with a rocker arm (306) that is rotatably adjustable about a longitudinal axis (LA) of the stabilization assembly (300) and about a transverse axis (TA2).

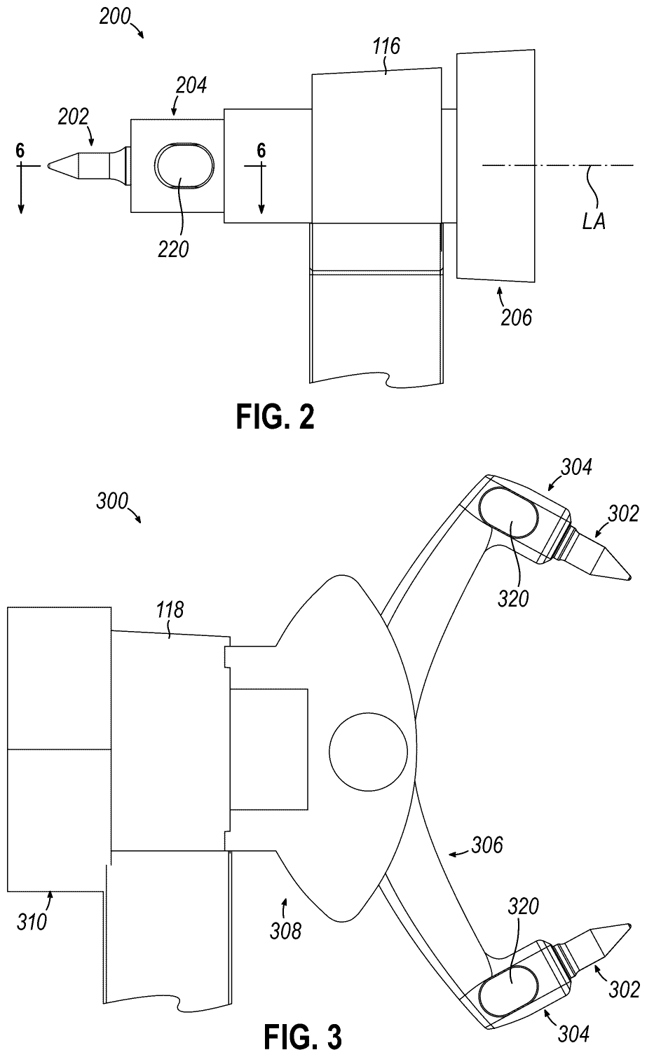

FIG. 2 illustrates the stabilization assembly (200), which connects with an upper portion (116) of arm (104). Stabilization assembly (200) includes a stabilization feature in the form of a pin (202), a receptacle (204) for selectively retaining the pin (202), and a force applicator in the form of a torque screw (206). Pin (202) is selectively retained in receptacle (204) via a retainer (220). In the present version, retainer (220) may be selectively depressed by an operator to thereby allow translation and thus release the pin (202) from the receptacle (204). Note that in other versions, some of which are shown and described herein, the retainer is not configured to be depressed or actuated by direct contact with a user. Receptacle (204) may also be referred to as a pin mount or pad mount in versions where pads replace pins.

FIG. 3 illustrates the stabilization assembly (300), which connects with an upper portion (118) of arm (106). Stabilization assembly (300) includes a pair of stabilization features in the form of pins (302), and a pair of receptacles (304) for selectively retaining the pins (302). As shown, the pair of receptacles (304) are part of a rocker arm (306), sometimes referred to in the present example as a two-pin rocker arm. A holding arch assembly (308) of the stabilization assembly (300) connects with the rocker arm (306) at one end while the other end of the holding arch assembly (308) includes an adjuster (310) that can be used to change a rotational position of the stabilization assembly (300) such that the rocker arm (306) with pins (302) is rotatably adjustable about a longitudinal axis (LA) of the stabilization assembly (300). Each of pins (302) is selectively retained in respective receptacles (304) via a respective retainer (320). Each of retainers (320) may be selectively depressed by an operator to thereby permit translation and thus release the respective pin (302) from the respective receptacle (304). Note that in other versions, some of which are shown and described herein, the retainer is not configured to be depressed or actuated by direct contact with a user.

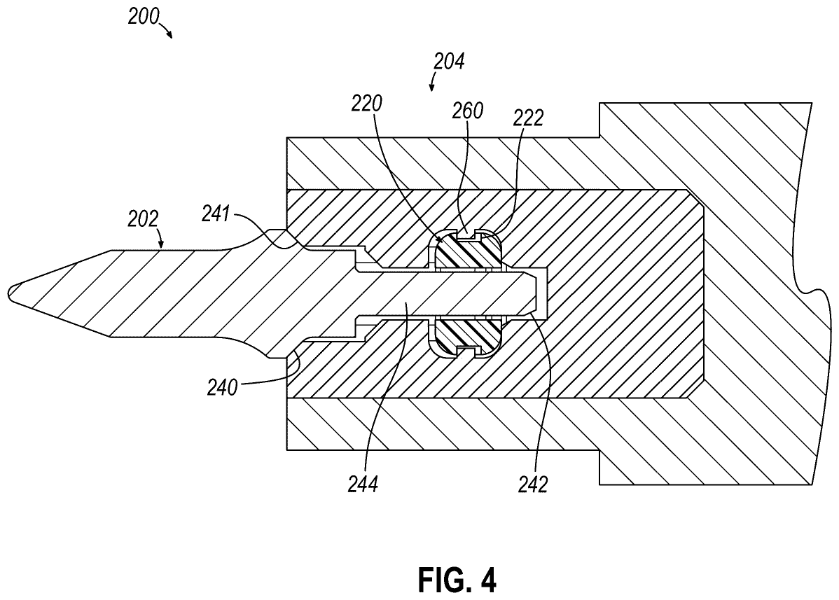

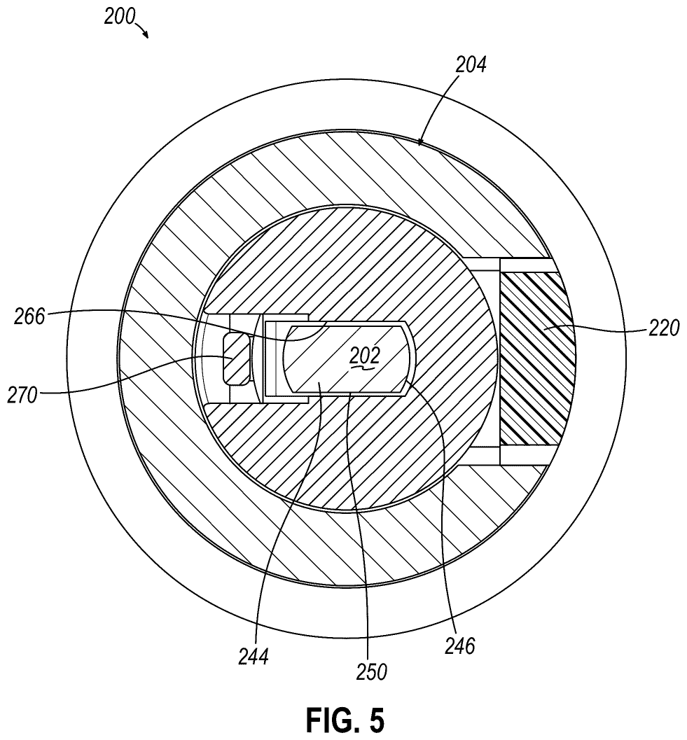

FIG. 4 illustrates a cross section view of the stabilization assembly (200). Pin (202) is shown having a tapered surface (240) complementary to a tapered surface (241) of receptacle (204) which centers pin (202). Pin (202) includes another tapered surface (242) at a proximal end of pin (202) to ease initial insertion into receptacle (204) and to apply a translating force onto retainer (220), as described in more detail below. Retainer (220) includes a slot (222) to engage a receptacle protrusion (260). Receptable protrusion (260) is slidably positioned within slot (222) to thereby be captured during translation of the retainer (220) within receptacle (204). As described in increased detail below and in FIG. 5, pin (202) includes a pin key (244) to prevent or inhibit rotation of pin (202) relative to receptacle (204) and retainer (220) about a longitudinal axis (LA) of pin (202). However, in some other versions pin key (244) may be omitted or modified to allow for rotation of pin (202) relative to receptacle (204) while the retainer (220), receptacle (204) and pin (202) remain configured to selectively lock or secure the translational movement of pin (202) relative to the receptacle (204). In other words, in these such other versions, the pin (202) is permitted to rotate yet remains secured in place from translational movement.

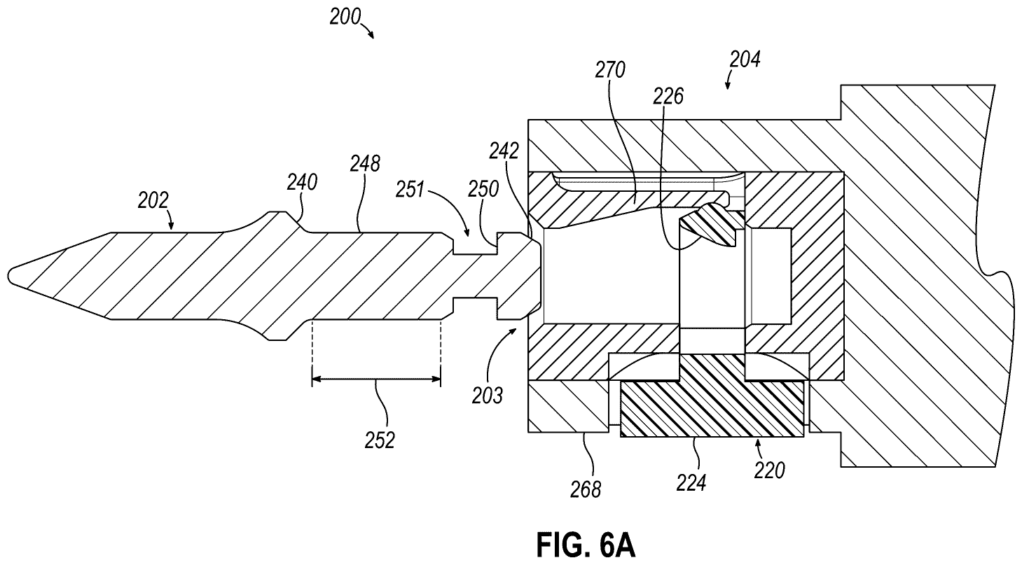

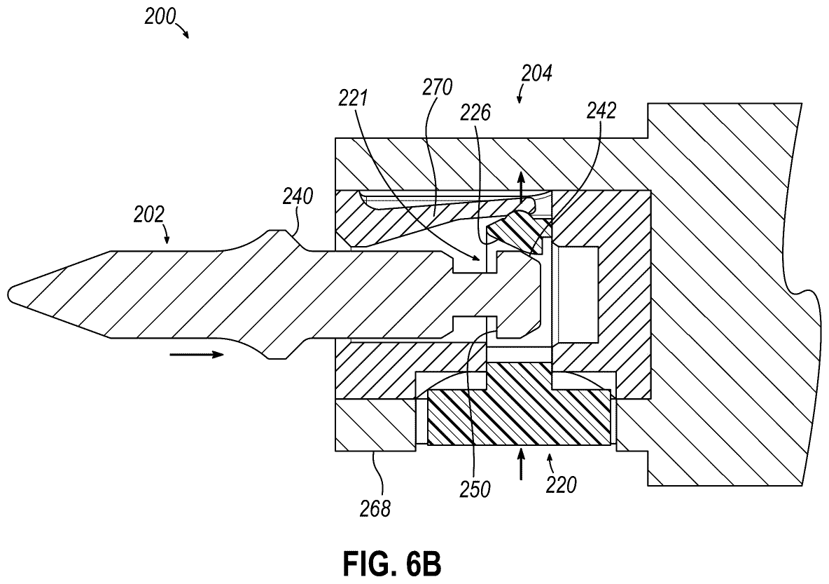

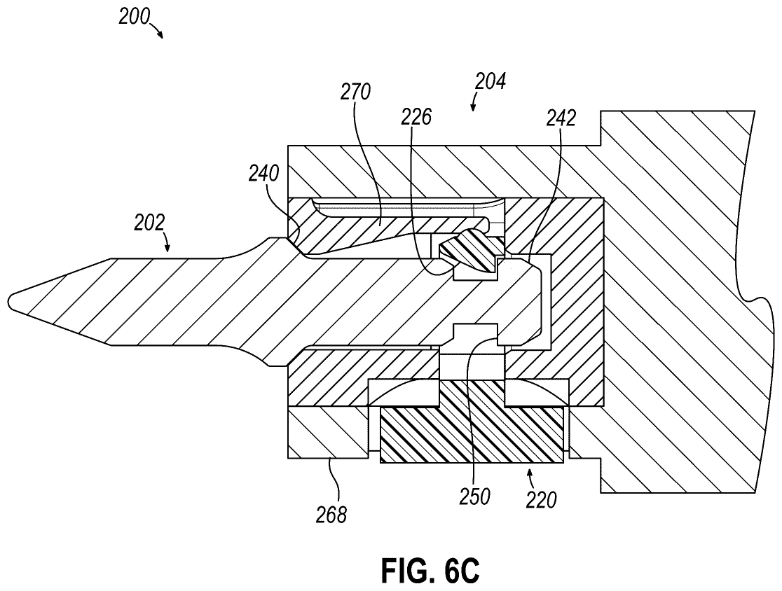

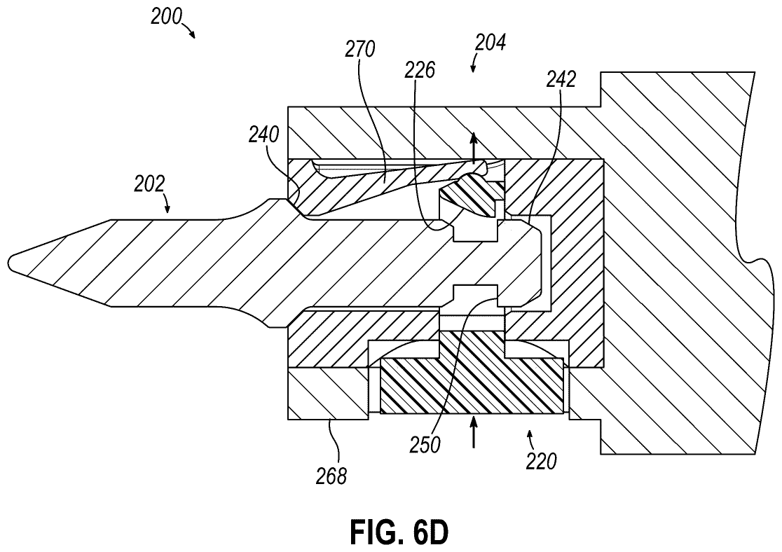

FIG. 5 illustrates another cross section view of the stabilization assembly (200). Pin key (244) includes a first pin profile (246) which matches and is slidable within a first receptacle profile (266) of receptacle (204). First receptacle profile (266) may also be referred to as a keyed profile. A portion of first pin profile (246) includes a pin diameter (248) as shown in FIG. 6A. As described below in detail, receptacle (204) includes a resilient feature or spring (270) to bias retainer (220) against pin (202) to thereby engage or couple pin (202) to receptacle (204).

FIGS. 6A-6C illustrate additional cross section views of stabilization assembly (200) with pin (202) being inserted and engaged with receptacle (204) via retainer (220). FIG. 6A illustrates pin (202) aligned with an opening (203) of receptacle (204) prior to insertion. With pin (202) outside of receptacle (204), spring (270) biases retainer (220) towards a closed position. Retainer (220) includes a surface (224) which may be recessed from immediately adjacent receptacle surface (268). Surface (224) is sized such that an operator may press surface (224) to thereby translate retainer (220) towards spring (270). As mentioned above, retainer (220) may be slidably captured within receptacle (204) using receptacle protrusion (260) and slot (222). Spring (270) is shown as a cantilever spring but may be any spring or resilient structure understood to a person of ordinary skill in the art such as a leaf spring, a coil spring, a resilient extension, a resilient element, etc. Retainer (220) includes a lip (226) for sliding against tapered surface (242) of pin (202) and for engaging with a pin step (250) of pin (202). The term “lip” is understood to be interchangeable with the term “chamfer” or “bevel.” Pin (202) includes a pin shaft length (252) sized to extend pin step (250) to lip (226) while tapered surface (240) of pin (202) contacts tapered surface (241) of receptacle (204).

FIG. 6B illustrates a cross section view of stabilization assembly (200) with pin (202) advanced inside receptacle (204) to where tapered surface (242) of pin (202) contacts lip (226). In the present example, lip (226) is configured as a chamfer, bevel, or ramp that may complement tapered surface (242) of pin (202). As pin (202) advances further inside receptacle (204), tapered surface (242) of pin (202) slides within an opening (221) of retainer (220) and against lip (226). This contact or engagement between pin (202) and retainer (220) causes retainer (220) to apply a force to spring (270) to ultimately overcome the force applied by spring (270). This action will drive retainer (220) in a direction perpendicular or transverse to the longitudinal axis (LA) of pin (202) and toward spring (270) which puts retainer (220) into an open position. Note that the transverse direction is shown generally by a transverse axis (TA) of FIG. 1.

FIG. 6C illustrates a cross section view of stabilization assembly (200) with pin (202) further advanced inside receptacle (204) to where retainer lip (226) has been forced inside of pin step (250) by spring (270). Pin (202) is now selectively engaged with receptacle (204) via engagement with retainer (220), which is engaged with receptacle (204) as described above based on engagement between receptacle protrusion (260) and slot (222) of retainer (220). This further puts the retainer (220) in the closed position. Upon pin (202) becoming selectively engaged with receptacle (204) and retainer (220), feedback may be provided to an operator to confirm the engaging or coupling. This feedback may be provided as audible feedback, where retainer (220) clicks or snaps when returning to the closed position; as tactile feedback, where an operator may feel that retainer (220) has returned to the closed position; or as visual feedback, where retainer (220) may visually indicate to an operator an open or closed position of the retainer (220) based on where surface (224) or retainer (220) is relative to surface (268) of receptacle (204).

FIG. 6D illustrates a cross section view of stabilization assembly (200) for removal of pin (202) from receptacle (204). An operator applies a force against surface (224) to thereby overcome the force from spring (270) and thus drive lip (226) away from and outside of pin step (250). Retainer (220) is thus shown in the open position. Pin (202) is then free to translate out of receptacle (204) along longitudinal axis (LA) of stabilization assembly (200). As shown in FIGS. 6A-6D, pin (202) includes a recess (251), best seen in FIG. 6A, that extends distally toward a tip of pin (202) from pin step (250). In the present version recess (251) is an annular recess that extends circumferentially about a longitudinal axis (LA) of pin (202). In the present example, lip (226) can be sized to closely match the dimensions of recess (251).

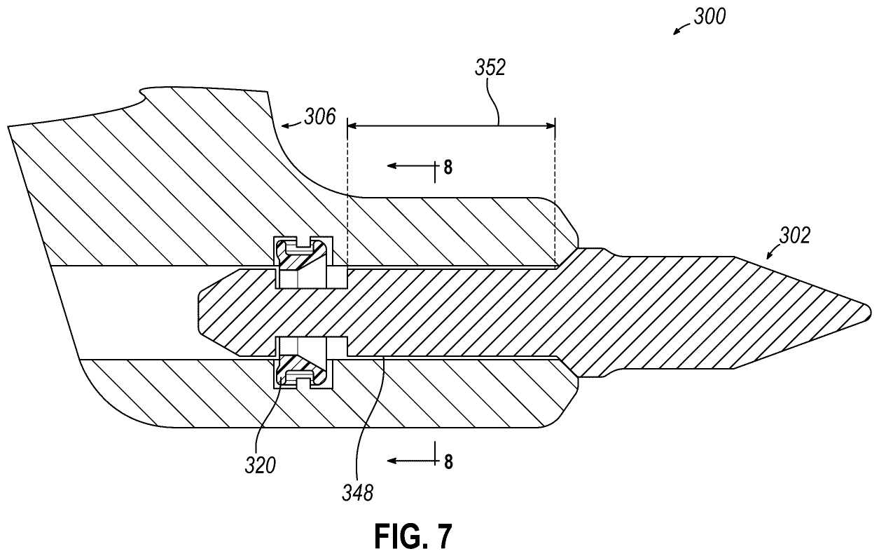

FIG. 7 illustrates a cross section view of stabilization assembly (300). Stabilization assembly (300) may be similar to stabilization assembly (200) except for some differences. For example, pin (302) includes a pin shaft length (352) and a pin diameter (348), each of which differ from the pin shaft length (252) and pin diameter (248) of pin (202) as shown in FIG. 6A. Further, pin (302) may be without a pin key similar to pin key (244) such that pin (302) may rotate within receptacle (304). These differences result in a poka-yoke design where pins (302) are prevented from engaging or coupling with receptacle (204). Such a configuration may be desired in stabilizations that use pins with differences such as differing pin geometries, angles, etc., as disclosed in U.S. Pat. No. 12,090,000, entitled “Head Stabilization Device with Non-Uniform Pins,” issued Sep. 17, 2024, herein incorporated by reference.

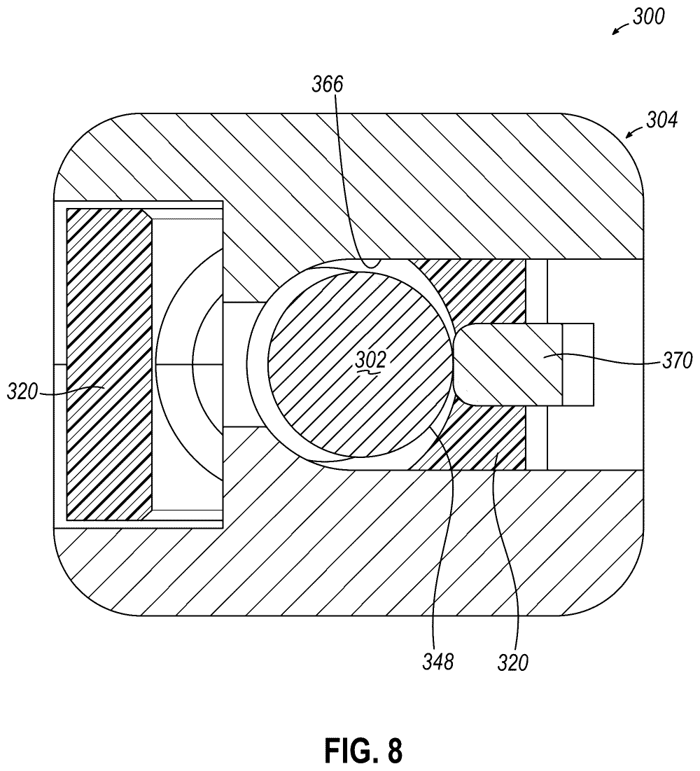

FIG. 8 illustrates another cross section view of stabilization assembly (300). Retainer (320) is shown engaged with pin (302) via a biasing force from spring (370). Pin (302) includes pin diameter (348) matching a second receptacle profile (366) of receptacle (304). Accordingly, the matching profiles of pin (302) and receptacle (304) differ from the matching profiles of pin (202) and receptacle (204). Again, this contributes to the poka-yoke design or configuration where the pins are matched to their specific receptacles of the respective stabilization assemblies.

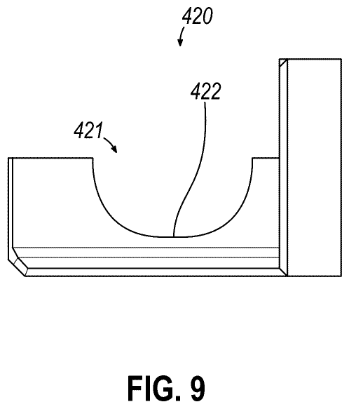

FIG. 9 illustrates an alternative retainer (420) that can be used in some versions in place of retainer (320) or retainer (220). Retainer (420) forms a partial opening (421) in the shape of a half circle from the side view. This is compared to the full circular shaped opening of retainer (320) and elongated circular shaped opening of retainer (220). With the structure or configuration of the partial opening (421) of retainer (420), the surface (422) of the opening (421) of retainer (420) is still able to engage with an inserted pin to lock or secure the pin relative to the receptacle. The main difference here compared to retainers (220, 320) is that a fully enclosed opening like those with retainers (220, 320) is not required in all versions.

### B. Alternate Exemplary Stabilization Assembly

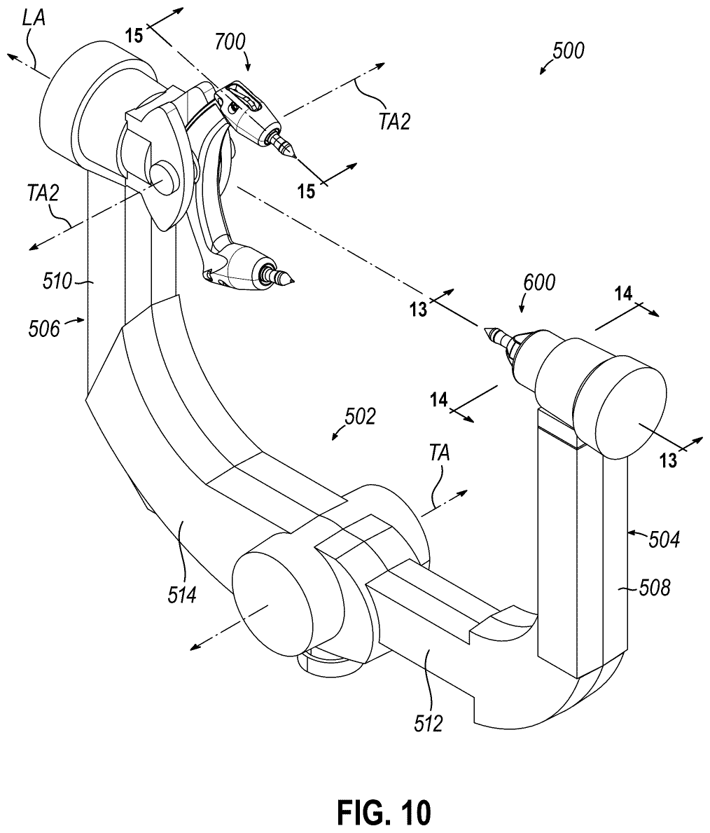

FIG. 10 illustrates another exemplary stabilization device in the form of a skull clamp (500). The skull clamp (500) includes a frame (502) comprised of a pair of arms (504, 506). The arms (504, 506) are moveable relative to one another to adjust a width or opening between upright portions (508, 510) of the arms (504, 506). A base of the frame (502) is comprised of lateral portions (512, 514) of the respective arms (504, 506). The skull clamp (500) is connectable with other equipment directly or indirectly such as a base unit (not shown) that connects with an operating table (not shown). As shown, the skull clamp (500) also includes stabilization assemblies (600, 700), each located at the upper portion of one of the arms (504, 506). Stabilization assembly (700) is configured with a rocker arm (706) that is rotatably adjustable about a longitudinal axis (LA) of the stabilization assembly (700) and about a transverse axis (TA2).

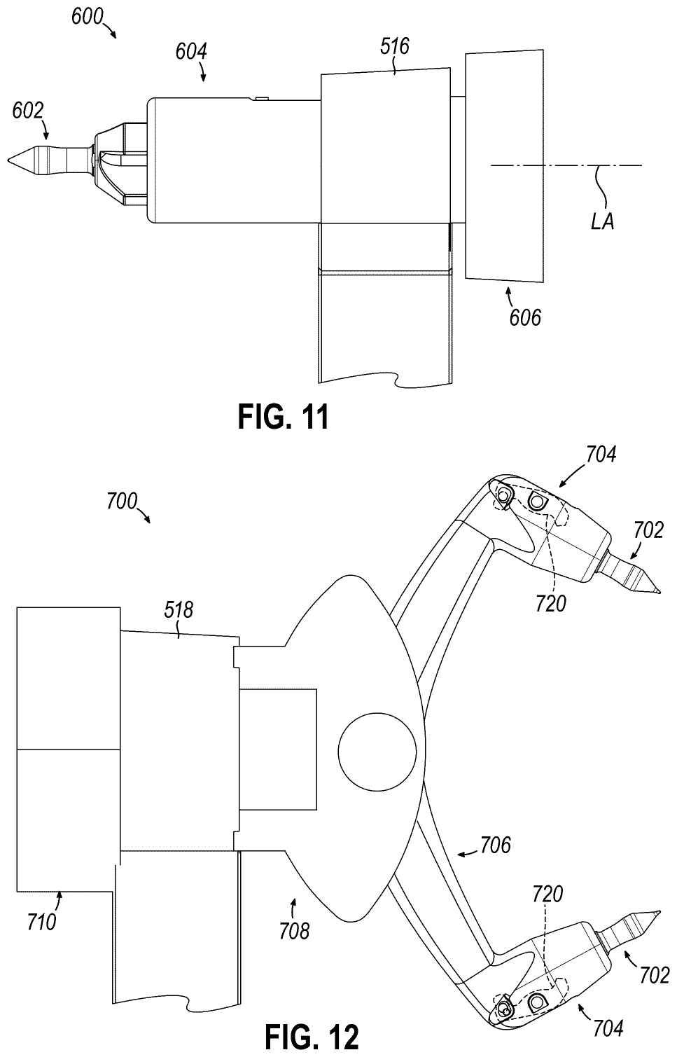

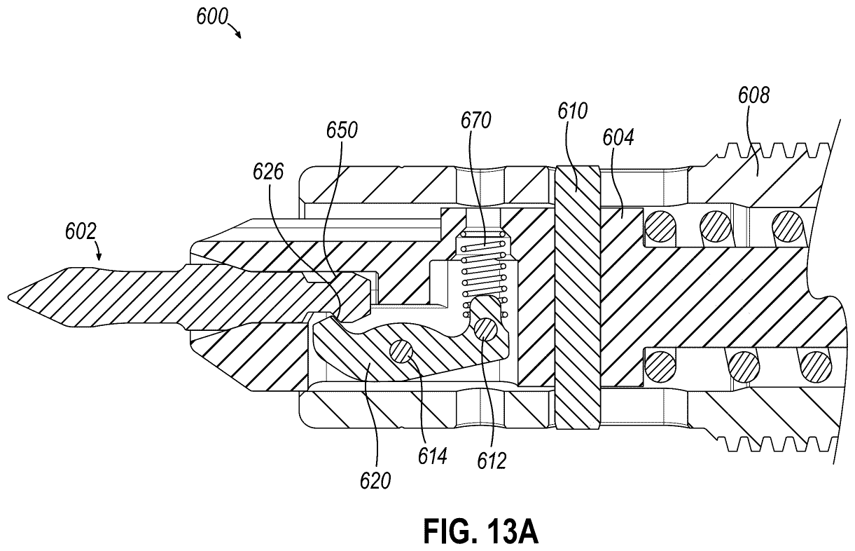

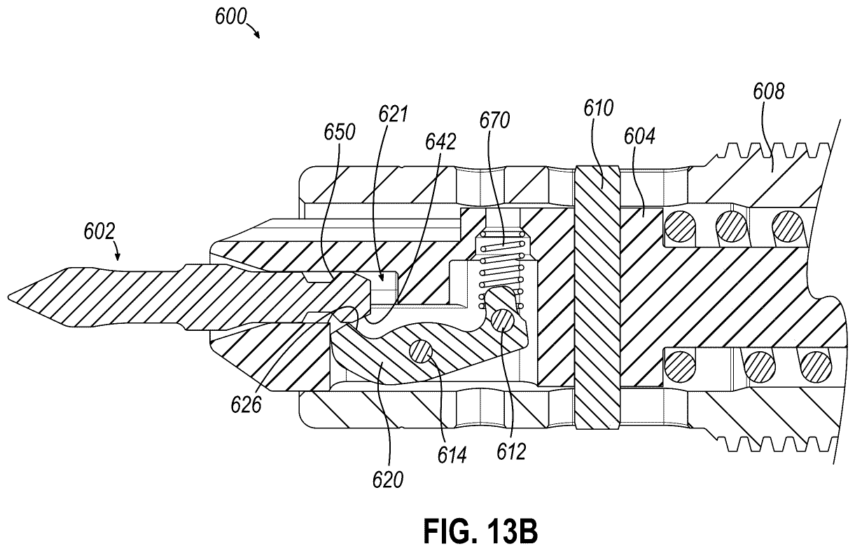

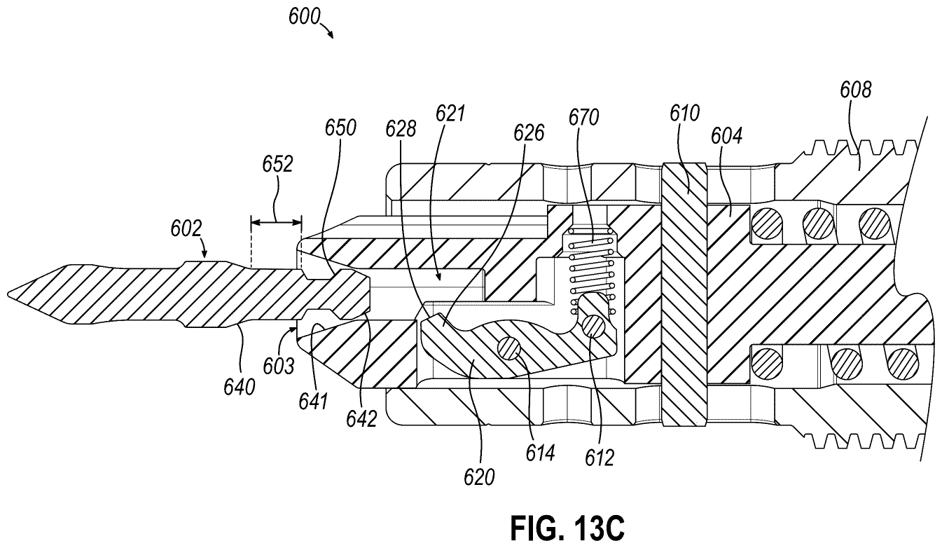

FIG. 11 illustrates the stabilization assembly (600), which connects with an upper portion (516) of arm (504). Stabilization assembly (600) includes a stabilization feature in the form of a pin (602), a receptacle (604) for selectively retaining the pin (602), and a force applicator in the form of a torque screw (606). Pin (602) is selectively retained in receptacle (604) via a retainer (620) best seen in FIGS. 13A-13C. When the pin (602) is pulled from the receptacle (604) by the operator, the pin (602) translates the retainer (620) such that the retainer (620) pivots and releases the pin (602) for removal from the receptacle (604). Receptacle (604) may also be referred to as a pin mount or pad mount in versions where pads replace pins.

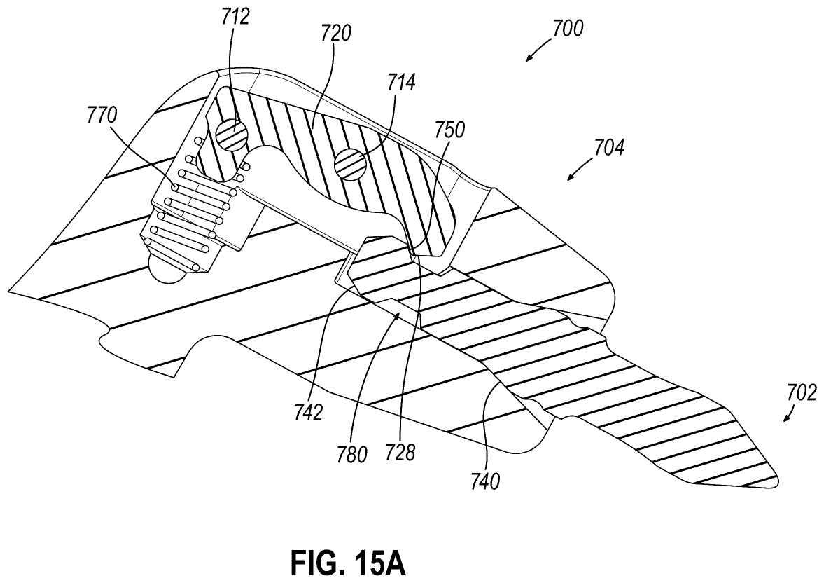

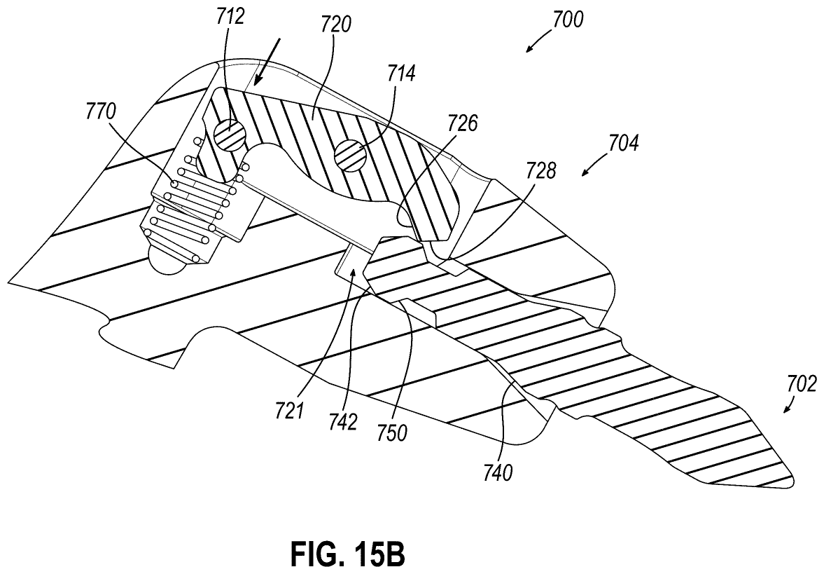

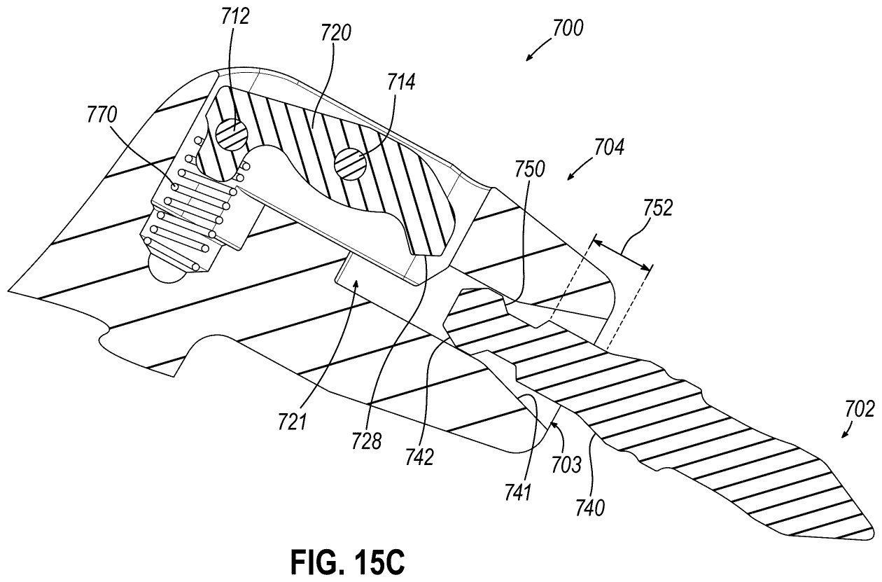

FIG. 12 illustrates the stabilization assembly (700), which connects with an upper portion (518) of arm (506). Stabilization assembly (700) includes a pair of stabilization features in the form of pins (702), and a pair of receptacles (704) for selectively retaining the pins (702). As shown, the pair of receptacles (704) are part of a rocker arm (706), sometimes referred to in the present example as a two-pin rocker arm. A holding arch assembly (708) of the stabilization assembly (700) connects with the rocker arm (706) at one end while the other end of the holding arch assembly includes an adjuster (710) that can be used to change a rotational position of the stabilization assembly (700) such that the rocker arm (706) with pins (702) is rotatably adjustable about a longitudinal axis (LA) of the stabilization assembly (700). Each of pins (702) is selectively retained in respective receptacles (704) via a respective retainer (720) best seen in FIGS. 15A-15C. When the pins (702) are pulled from their respective receptacles (704) by the operator, the pins (702) translate the respective retainers (720) such that the respective retainers (720) translate and releases the pins (702) from the respective receptacles (704).

FIGS. 13A-13C illustrate additional cross section views of stabilization assembly (600) with pin (602) being inserted and engaged with receptacle (604) via retainer (620), or conversely being removed and disengaged with receptacle (604). In addition to receptacle (604), stabilization assembly (600) includes torque screw housing (608), and receptacle (604) is connected with torque screw housing (608) by way of a pinned connection via post (610) in the present example.

Within receptacle (604) resides retainer (620) and a spring (670). Spring (670) is positioned and configured to contact retainer (620) such that retainer (620) can pivot about a pinned connection with receptacle (604) based on influence from spring (670) or influence from other sources that overcome the spring force imparted by spring (670). In the present example, a post (612) extends through retainer (620) and spring (670) contacts post (612) on one end of spring (670) while the opposite end of spring (670) contacts and interior surface of receptacle (604). In the present example, but not required in all versions, receptacle (604) includes oversized bores (not shown) that are configured to receive the ends of post (612). Because the bores are oversized, post (612) is able to move relative to receptacle (604) when retainer (620) pivots. In some other versions, post (612) may terminate within receptacle (604) such that the oversized bores are not required.

As mentioned, retainer (620) is configured to pivot, and in doing so moves from an open state or position to a closed state or position and vice versa. To achieve the pivoting action, retainer (620) has a pinned connection with receptacle (604). Specifically, post (614) extends through a bore in retainer (620) and terminates on each end within receptacle (604). As mentioned above, and further discussed below, the pivoting action of retainer (620) is caused by either the spring force from spring (670) or an externally applied force that overcomes the spring force. Retainer (620) also has another lateral support post or guidepost (not shown) extending therethrough. The lateral support post terminates within the interior of receptacle (604) such that the lateral support post does not engage with receptacle (604). In this manner, the guidepost provides lateral stability to retainer (620) without interfering with the ability for retainer (620) to pivot.

In FIG. 13A pin (602) is fully advanced within receptacle (604). In this state, spring (670) within receptacle (604) biases retainer (620) such that a sloped surface (626) of retainer (620) engages or contacts a tapered surface (650) of pin (602). In this state or position, pin (602) is selectively engaged with receptacle (604) via engagement with retainer (620). More specifically, with pin (602) fully inserted, force applied by spring (670) upon retainer (620) pivots retainer (620) to a closed state or position where sloped surface (626) of retainer (620) contacts and engages with tapered surface (650) of pin (602). As mentioned above, in the present example retainer (620) is pivotably captured within receptacle (604) by post (614). Upon pin (602) becoming selectively engaged with receptacle (604) and retainer (620), feedback may be provided to an operator to confirm the engaging. This feedback may be provided as audible feedback, where retainer (620) clicks or snaps when returning to the closed position; or as tactile feedback, where an operator may feel that retainer (620) has returned to the closed position.

FIG. 13B illustrates a cross section view of stabilization assembly (600) with pin (602) partially advanced within receptacle (604) to where a tapered surface (642) of pin (602) is just past a sloped surface (628) of retainer (620). In the present example, sloped surface (628) is configured as a chamfer, bevel, or ramp that complements tapered surface (642) of pin (602). As pin (602) advances within receptacle (604), proximal portion of pin (602) slides within an opening or space (621) within receptacle (604) as tapered surface (642) of pin (602) advances past sloped surface (628) of receptacle (620). In the process of pin (602) advancing, the contact or engagement between tapered surface (642) of pin (602) and sloped surface (628) of retainer (620) causes retainer (620) to pivot and in doing so to apply a force to spring (670) to ultimately overcome the force applied by spring (670) thereby compress spring (670). This action will drive retainer (620) in a direction perpendicular or transverse to the longitudinal axis (LA) of pin (602) and toward spring (670) which puts retainer (620) into an open state or position as shown in FIG. 13B. Note that the transverse direction is shown generally by a transverse axis (TA) of FIG. 10. With retainer (620) pivoted downward to the open state or position, pin (602) can continue to advance within receptacle (604) to full seat within receptacle (604).

It should also be observed with respect to FIG. 13B that removal of pin (602) works in a similar manner with respect to the interaction between pin (602) and retainer (620). More specifically, when removing pin (602) by moving pin (602) from the position shown in FIG. 13A to the position shown in FIG. 13B, it is the interaction between tapered surface (650) of pin (602) and sloped surface (626) of retainer (620) that drives retainer (620) from the closed state to the open state by pivoting retainer (620) downward and compressing spring (670). In this manner, it should be noted that pin (602) has dual tapered surfaces (642, 650) and retainer (620) has dual sloped surfaces (626, 628), and it is the interaction among these pairs of surfaces that allows for pin (602) to cause pivoting of retainer (620) to overcome the force of spring (670) when either inserting pin (602) within receptacle (604) or removing pin (602) from receptacle (604).

FIG. 13C illustrates pin (602) aligned with an opening (603) of receptacle (604). This position could be achieved either when removing pin (602) from receptacle (604) or when inserting pin (602) within receptacle (604). In the case of pin removal, as mentioned above, by pulling pin (602) away from receptacle (604), tapered surface (650) of pin (602) interacts with sloped surface (626) of retainer (620) such that the retainer (620) pushes against the force of the spring (670) to move into the open position, thereby allowing removal of the pin (602). With pin (602) outside of receptacle (604), spring (670) biases retainer (620) towards a closed position as shown in FIGS. 13A and 13C.

In the present example, spring (670) is shown as a coil spring but may be any spring or resilient structure understood to a person of ordinary skill in the art such as a leaf spring, a cantilever spring, a resilient extension, a resilient element, etc. Additionally, retainer (620) includes sloped surfaces (626, 628) for sliding against tapered surfaces (642, 650) of pin (602). The term “sloped surfaces” and/or “tapered surfaces” are understood to be interchangeable with the terms “chamfer” or “bevel,” or other terms that will be apparent to those of ordinary skill in the art in view of the teachings herein.

Also with respect to FIGS. 13A-13C, pin (602) includes a pin shaft length (652) sized to extend tapered surface (650) of pin (602) to sloped surface (626) of retainer (620) while tapered surface (640) of pin (602) contacts tapered surface (641) of receptacle (604). The combination of simultaneous contact or engagement between pin (602) and receptacle (604) on the one hand, and pin (602) and retainer (620) on the other hand is another aspect that provides for a poka-yoke configuration where pins are specifically designed to fit within a specific receptacle. In this configuration, the pin has a first portion that includes a tapered surface that reduces in diameter as the tapered surface extends proximally, and the tapered surface is configured to contact a corresponding tapered surface of the stabilization device or receptacle to inhibit lateral movement of the pin.

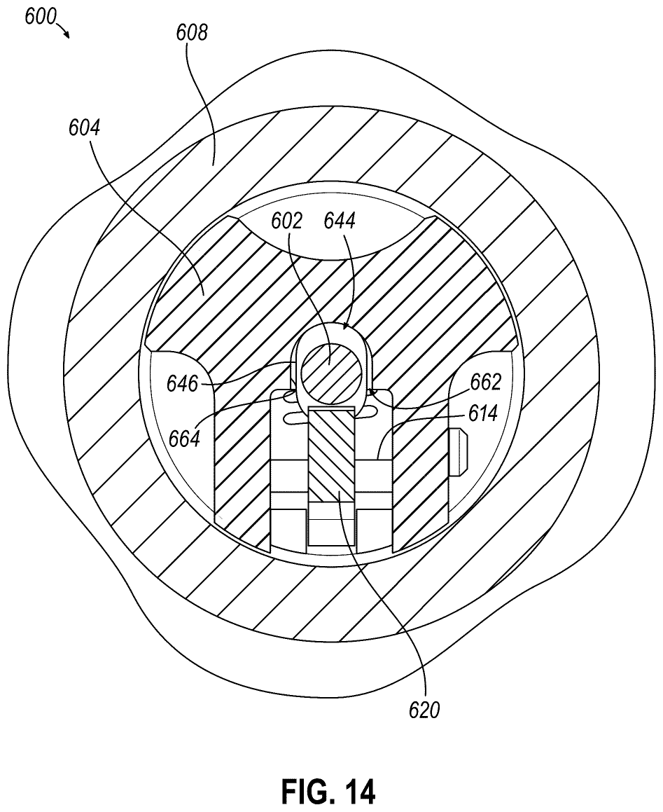

Referring to FIG. 14, another cross section of stabilization assembly (600) is shown illustrating a keyed configuration for pin (602) and receptable (604). More specifically, pin (602) includes a pin key (644) to prevent or inhibit rotation of pin (602) relative to receptacle (604) and retainer (620) about a longitudinal axis (LA) of pin (602). As shown in FIG. 14, pin key (644) includes a first pin profile (646) which matches in shape and is slidable within a keyhole (662) of receptacle (604), the keyhole (662) including a first receptacle profile (664). First pin profile (646) may also be referred to as a pin key profile, while first receptacle profile (664) may also be referred to as a receptacle keyed profile. Moreover, these profiles are corresponding like with a key and keyhole relationship where the pin key (644) is configured to fit within the keyhole (662) and the keyhole (662) is likewise configured to receive the pin key (644). However, in some other versions pin key (644) or keyhole (662) may be omitted or modified to allow for rotation of pin (602) relative to receptacle (604) while the retainer (620), receptacle (604) and pin (602) remain configured to selectively lock or secure the translational movement of pin (602) relative to the receptacle (604). In other words, in these such other versions, the pin (602) is permitted to rotate yet remains secured in place from translational movement.

In the present example, pin (702) and pin (602) differ in at least the respect that pin (602) has the keyed configuration where pin key (644) includes first pin profile (646) having planar portions, planar surfaces, or flat sides and keyhole (662) of receptacle (604) includes first receptacle profile (664) having complementary or corresponding planar portions, planar surfaces, or flat sides such that rotatability of pin (602) within receptacle (604) is restricted or prevented when these flat sides of the pin (602) and receptacle (604) are adjacent to one another or in contact or substantial contact with one another. In this manner, these planar portions, planar surface, or flat sides of the pin (602) are considered part of the pin key configuration and profile while the planar portions, planar surface, or flat sides of the receptacle (604) are considered part of the receptacle keyhole configuration and profile. With pin (702) the circular shape and complementary or corresponding circular shape for the profile of receptacle (704) does not restrict or prevent pin (702) from being able to rotate relative to receptacle (704). Furthermore, the keyed profile of receptacle (604) based on its first receptacle profile (664), represents a feature that provides a poka-yoke configuration where pin (602) can fit securely within receptacle (604) but pin (702) cannot fit securely within receptacle (604). In the present example, for instance, the proximal portion of pin (702) is too large to fit within receptacle (604). Consequently, a user is unable to mistakenly install pin (702) within receptacle (604). This can be particularly beneficial where different pin designs might be used within a stabilization such that not all pins are uniform. It is also noted here that while the present example illustrates planar portion, planar surface, or flat sides for the pin key and receptacle keyhole, other corresponding features could be used instead of or in addition to these to achieve the same result and such other corresponding features will be apparent to those of ordinary skill in the art in view of the teachings herein.

FIGS. 15A-15C illustrate additional cross section views of stabilization assembly (700) with pin (702) being inserted and engaged with receptacle (704) via retainer (720). Overall, the way pin (702) selectively engages with retainer (720) in the same manner as above with respect to pin (602) and retainer (620). For instance, similar to the illustration and description of FIG. 13A, FIG. 15A illustrates a cross section view of stabilization assembly (700) with pin (702) further advanced inside receptacle (704) to where a distal end of retainer having sloped surface (728) is located within a recess or groove (780) of pin (702). Like above, in this fully inserted state, spring (770) is biasing retainer (720) to couple with or engage with recess (780) of pin (702) thereby selectively retaining pin (702) within receptacle (704).

As with receptacle (604) discussed above, within receptacle (704) resides retainer (720) and a spring (770). Spring (770) is positioned and configured to contact retainer (720) such that retainer (720) can pivot about a pinned connection with receptacle (704) based on influence from spring (770) or influence from other sources that overcome the spring force imparted by spring (770). In the present example, a post (712) extends through retainer (720) and spring (770) contacts post (712) on one end of spring (770) while the opposite end of spring (770) contacts and interior surface of receptacle (704). In the present example, but not required in all versions, receptacle (704) includes oversized bores (not shown) that are configured to receive the ends of post (712). Because the bores are oversized, post (712) is able to move relative to receptacle (704) when retainer (720) pivots. In some other versions, post (712) may terminate within receptacle (704) such that the oversized bores are not required.

As with retainer (620) discussed above, retainer (720) is configured to pivot, and in doing so moves from an open state or position to a closed state or position and vice versa. To achieve the pivoting action, retainer (720) has a pinned connection with receptacle (704). Specifically, post (714) extends through a bore in retainer (720) and terminates on each end within receptacle (704). The pivoting action of retainer (720) is caused by either the spring force from spring (770) or an externally applied force that overcomes the spring force. Retainer (720) also has another lateral support post (not shown) extending therethrough. This lateral support post or guidepost terminates within the interior of receptacle (704) such that the lateral support post does not engage with receptacle (704). In this manner, the guidepost provides lateral stability to retainer (720) without interfering with the ability for retainer (720) to pivot.

FIG. 15B illustrates a cross section view of stabilization assembly (700) with pin (702) partially advanced within receptacle (704) to where a tapered surface (742) of pin (702) is just past a sloped surface (728) of retainer (720). In the present example, sloped surface (728) is configured as a chamfer, bevel, or ramp that complements tapered surface (742) of pin (702). As pin (702) advances within receptacle (704), proximal portion of pin (702) slides within an opening or space (721) within receptacle (704) as tapered surface (742) of pin (702) advances past sloped surface (728) of receptacle (720). In the process of pin (702) advancing, the contact or engagement between tapered surface (742) of pin (702) and sloped surface (728) of retainer (720) causes retainer (720) to pivot and in doing so to apply a force to spring (770) to ultimately overcome the force applied by spring (770) thereby compressing spring (770). This action will drive retainer (720) in a direction perpendicular or transverse to the longitudinal axis (LA) of pin (702) and toward spring (770) which puts retainer (720) into an open state or position as shown in FIG. 15B. Note that the transverse direction is shown generally by a transverse axis (TA) of FIG. 10. With retainer (720) pivoted downward to the open state or position, pin (702) can continue to advance within receptacle (704) to full seat within receptacle (704).

It should also be observed with respect to FIG. 15B that removal of pin (702) works in a similar manner with respect to the interaction between pin (702) and retainer (720). More specifically, when removing pin (702) by moving pin (702) from the position shown in FIG. 15A to the position shown in FIG. 15B, it is the interaction between tapered surface (750) of pin (702) and sloped surface (726) of retainer (720) that drives retainer (720) from the closed state to the open state by pivoting retainer (720) downward and compressing spring (770). In this manner, it should be noted that pin (702) has dual tapered surfaces (742, 750) and retainer (720) has dual sloped surfaces (726, 728), and it is the interaction among these pairs of surfaces that allows for pin (702) to cause pivoting of retainer (720) to overcome the force of spring (770) when either inserting pin (702) within receptacle (704) or removing pin (702) from receptacle (704).

FIG. 15C illustrates pin (702) aligned with an opening (703) of receptacle (704). This position could be achieved either when removing pin (702) from receptacle (704) or when inserting pin (702) within receptacle (704). In the case of pin removal, as mentioned above, by pulling pin (702) away from receptacle (704), tapered surface (750) of pin (702) interacts with sloped surface (726) of retainer (720) such that the retainer (720) pushes against the force of the spring (770) to move into the open position, thereby allowing removal of the pin (702). With pin (702) outside of receptacle (704), spring (770) biases retainer (720) towards a closed position as shown in FIGS. 15A and 15C.

It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A device for stabilizing a head of a patient in a medical procedure, wherein the device comprises:
(a) a frame; and
(b) a stabilization assembly connectable with the frame, wherein the stabilization assembly comprises:
(i) a receptacle including an opening;
(ii) a pin slidable within the opening of the receptacle; and
(iii) a retainer coupled to the receptacle and configured to selectively engage with the pin to selectively retain the pin within the receptacle.

2. The device of claim 1, wherein the retainer is configured to provide feedback to an operator once the pin is engaged with the retainer.

3. The device of claim 1, wherein the retainer is configured to transition between an open position and a closed position, wherein the retainer is biased towards the closed position.

4. The device of claim 3, wherein the retainer includes a surface with chamfer configured to slide against the pin to transition the retainer from the closed position to the open position.

5. The device of claim 3, wherein the pin includes a surface with a chamfer configured to slide against the retainer to thereby transition the retainer from the closed position to the open position.

6. The device of claim 3, wherein with the pin fully seated within the opening in the receptacle, the retainer is configured to transition from the closed position to the open position by applying a force to the pin in a direction opposite to a direction for inserting the pin within the opening of the receptacle.

7. The device of claim 1, wherein the pin includes a step along a longitudinal axis, wherein the retainer is configured to selectively engage with the step of the pin.

8. The device of claim 1, wherein the receptacle includes a tapered surface, wherein the pin includes a tapered surface, wherein the tapered surface of the receptacle is configured to engage with the tapered surface of the pin to inhibit lateral movement of the pin while the pin is engaged with the retainer.

9. The device of claim 1, wherein the receptacle includes a spring configured to apply a force on the retainer to thereby engage the retainer to the pin.

10-11. (canceled)

12. The device of claim 1, wherein the opening of the receptacle includes a keyhole configured to selectively receive a corresponding keyed portion of the pin once the pin is within the opening of the receptacle.

13. (canceled)

14. The device of claim 1, wherein the retainer is configured to inhibit intentional longitudinal translation of the pin while the retainer is engaged with the pin.

15. The device of claim 1, wherein the frame comprises a pair of arms each with an upright portion and a lateral portion, wherein the stabilization assembly is connectable with a select one of the upright portions of the frame.

16. The device of claim 15, wherein the pair of arms are adjustable relative to one another to adjust a width or spacing between the upright portions.

17. The device of claim 1, further comprising a second stabilization assembly, wherein each of the stabilization assembly and the second stabilization assembly are connectable with the frame, wherein the pin is only connectable with the stabilization assembly but not with the second stabilization assembly such that the pin is not interchangeable among the stabilization assembly and the second stabilization assembly.

18-21. (canceled)

22. A pin for use with a stabilization device in a medical procedure to stabilize a portion of a patient, wherein the pin comprises a first portion configured to be received within the stabilization device, and a second portion configured to protrude from the stabilization device, wherein the first portion includes an engagement surface configured to selectively engage with the stabilization device to selectively retain the pin with the stabilization device.

23-29. (canceled)

30. The pin of claim 22, wherein a proximal end of the pin includes a chamfered surface.

31. The pin of 22, wherein the first portion includes a tapered surface that reduces in diameter as the tapered surface extends proximally, wherein the tapered surface is configured to contact a corresponding tapered surface of the stabilization device to inhibit lateral movement of the pin.

32. (canceled)

33. The pin of claim 22, wherein the pin comprises a pair of opposite facing flat surfaces and a pair of opposite facing curved surfaces.

34-35. (canceled)

36. A pin holder for use with a stabilization device in a medical procedure to stabilize a portion of a patient, wherein the pin holder is configured to receive a pin, wherein the pin holder comprises a receptacle with an opening for receiving the pin, wherein the pin holder comprises a resilient feature located within the receptacle.

37. The pin holder of claim 36, wherein the receptacle includes a tapered surface configured to engage with a complementary tapered surface of the pin to inhibit lateral movement of the pin.

38. The pin holder of claim 36, wherein the pin holder further comprises a retainer configured to transition between an open position and a closed position, wherein the retainer is biased towards the closed position by the resilient feature, wherein with the pin fully seated within the opening in the receptacle, the retainer is configured to transition from the closed position to the open position by applying a force to the pin in a direction opposite to a direction for inserting the pin within the opening of the receptacle.

39-44. (canceled)

45. The pin holder of any one of claim 38 through claim 44, wherein the opening of the receptacle includes a keyhole configured to selectively receive a corresponding keyed portion of the pin once the pin is within the opening of the receptacle.

46-50. (canceled)
